(19) United States
(12) Patent Application Publication
(10) Pub. No.: US 2026/0191516 A1
(43) Pub. Date: Jul. 9, 2026

(54) BIOPSY DEVICE WITH IN-LINE SAMPLE IMAGING OUTSIDE ASPIRATION PATH

(71) Applicant: Devicor Medical Products, Inc., Cincinnati, OH (US)

(72) Inventors: Jack A. RANDALL, Cincinnati, OH (US); Garrett A. HOUSEHOLDER, Cincinnati, OH (US); James O. ROGERS, III, Milford, OH (US); Andrew P. NOCK, Dayton, OH (US); Lyle JAMES, Kettering, OH (US)

(21) Appl. No.: 19/556,187

(22) Filed: Mar. 4, 2026

Related U.S. Application Data
(63) Continuation of application No. PCT/US2024/048123, filed on Sep. 24, 2024.
(60) Provisional application No. 63/540,965, filed on Sep. 28, 2023.

(51) Int. Cl. A61B 10/02; A61B 10/00

(52) U.S. Cl. CPC A61B 10/0266; A61B 10/0096; A61B 2010/0225

(57) ABSTRACT

A biopsy system includes a biopsy device, a control module, and a tissue transport tube. The biopsy device includes a probe, a needle, and a cutter. The needle extends distally from the probe. The cutter is movable relative to the needle to sever one or more tissue samples. The control module is in communication with the biopsy device and includes a sample tray configured to receive one or more tissue samples severed by the cutter. The tissue transport tube is adapted to connect between the tissue sample holder and the cutter of the biopsy device. The sample tray has a sampling configuration and an imaging configuration. The sample tray is configured to move between a sampling axis corresponding to the sapling configuration and an imaging axis corresponding to the imaging configuration to receive and subsequently image one or more tissue samples received within the sample tray.

### PRIORITY

This application claims priority to U.S. Provisional Application No. 63/540,965, entitled “Biopsy Device with In-Line Sample Imaging Outside Aspiration Path,” filed on Sep. 28, 2023, the disclosure of which is hereby incorporated by reference herein.

### BACKGROUND

Biopsy samples have been obtained in a variety of ways in various medical procedures using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, PEM guidance, BSGI guidance, or otherwise.

Merely exemplary biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,017,316, entitled “Vacuum Control System and Method for Automated Biopsy Device,” issued Jan. 25, 2000; U.S. Pat. No. 6,086,544, entitled “Control Apparatus for an Automated Surgical Biopsy Device,” issued Jul. 11, 2000; U.S. Pat. No. 6,432,065, entitled “Method for Using a Surgical Biopsy System with Remote Control for Selecting an Operational Mode,” issued Aug. 13, 2002; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 21, 2012; U.S. Pat. No. 8,206,316, entitled “Tetherless Biopsy Device with Reusable Portion,” issued Jun. 26, 2012; U.S. Pat. No. 8,491,496, entitled “Biopsy Device with Sample Storage,” issued on Jul. 23, 2013; U.S. Pat. No. 8,702,623, entitled “Biopsy Device with Discrete Tissue Chambers,” issued Apr. 22, 2014; U.S. Pat. No. 8,764,680, entitled “Handheld Biopsy Device with Needle Firing,” issued Jul. 1, 2014; U.S. Pat. No. 9,095,326, entitled “Biopsy System with Vacuum Control Module,” issued Aug. 4, 2015; U.S. Pat. No. 9,326,755, entitled “Biopsy Device Tissue Sample Holder with Bulk Chamber and Pathology Chamber,” issued May 3, 2016; U.S. Pat. No. 9,345,457, entitled “Presentation of Biopsy Sample by Biopsy Device,” issued May 24, 2016; U.S. Pat. No. 10,905,404, entitled “Tissue Sample Holder with Enhanced Features,” issued on Feb. 2, 2021; and U.S. Pat. No. 11,504,101, entitled “Biopsy Device with Remote Multi-Chamber Tissue Sample Holder” issued on Nov. 22, 2022. The disclosure of each of the above-cited U.S. patents is incorporated by reference herein.

Additional exemplary biopsy devices and biopsy system components are disclosed in U.S. Pat. Pub. No. 2006/0074345, entitled “Biopsy Apparatus and Method,” published Apr. 6, 2006, now abandoned; U.S. Pat. Pub. No. 2010/0152610, entitled “Hand Actuated Tetherless Biopsy Device with Pistol Grip,” published Jun. 17, 2010; U.S. Pat. Pub. No. 2010/0160819, entitled “Biopsy Device with Central Thumbwheel,” published Jun. 24, 2010, now abandoned; U.S. Pat. Pub. No. 2012/0283563, entitled “Biopsy Device with Manifold Alignment Feature and Tissue Sensor,” published Nov. 8, 2012, now abandoned; U.S. Pat. App. No. 2013/0150751, entitled “Biopsy Device With Slide-In Probe,” published Jun. 13, 2013; and U.S. Pat. App. No. 2013/0324882, entitled “Control for Biopsy Device,” published Dec. 5, 2013. The disclosure of each of the above-cited U.S. patent Application Publications, U.S. Non-Provisional patent applications, and U.S. Provisional patent applications is incorporated by reference herein.

While several systems and methods have been made and used for obtaining a biopsy sample, it is believed that no one prior to the inventor has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1A depicts a perspective view of an exemplary biopsy system;

FIG. 1B depicts a detailed perspective view of a needle of a biopsy device of the biopsy system of FIG. 1A;

FIG. 2 depicts a perspective view of an exemplary tissue handling assembly that may be readily incorporated into the system of FIG. 1;

FIG. 3A depicts a top plan view of a tissue sample holder of the tissue handling assembly of FIG. 2;

FIG. 3B depicts another top plan view of the tissue sample holder of FIG. 3A, the tissue sample holder advancing a tissue sample for imaging;

FIG. 4 depicts a partial perspective view of the tissue sample holder of FIG. 3A;

FIG. 5A depicts a perspective view of an exemplary alternative tissue sample holder that may be readily incorporated into the tissue handling assembly of FIG. 2, the tissue sample holder being in a transport configuration;

FIG. 5B depicts another perspective view of the tissue sample holder of FIG. 5A, the tissue sample holder being in an imaging configuration;

FIG. 5C depicts a front elevational view of the tissue sample holder of FIG. 5A, the tissue sample holder being in the imaging configuration of FIG. 5B;

FIG. 6 depicts a perspective view of another exemplary alternative tissue sample holder that may be readily incorporated into the tissue handling assembly of FIG. 2; and

FIG. 7 depicts a front elevational view of the tissue sample holder of FIG. 7 with a portion of the tissue sample holder being rotated for imaging of a tissue sample.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Overview of Exemplary Biopsy System

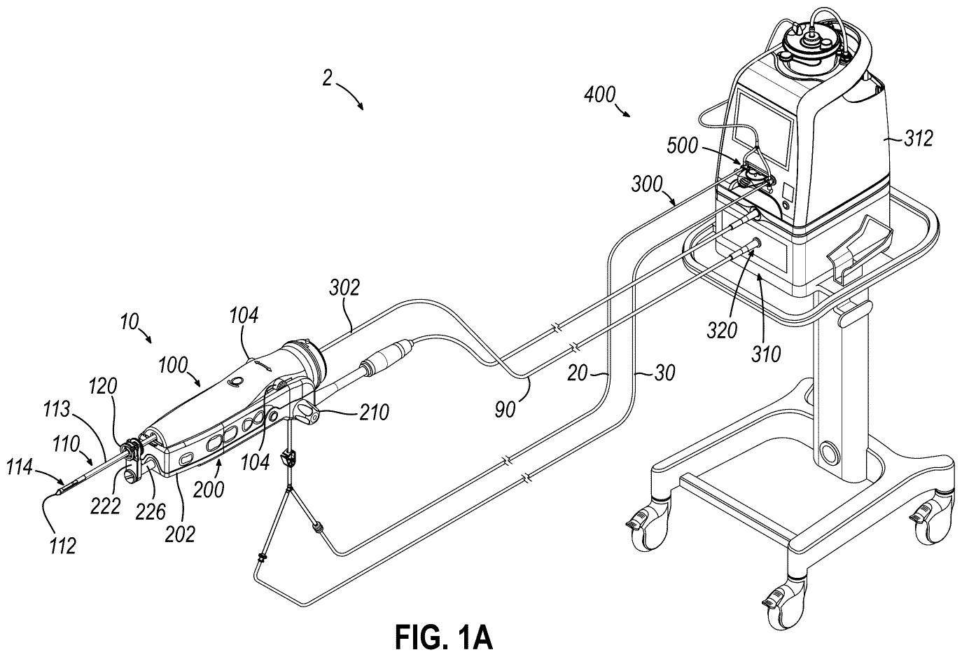

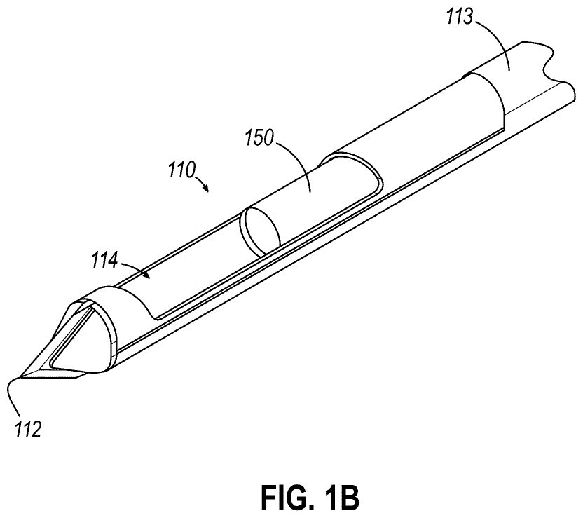

FIG. 1A depicts an exemplary biopsy system (2) including a biopsy device (10) and a vacuum control module (400). Biopsy device (10) of the present example includes a probe (100) and a holster (200). A needle (110) extends distally from probe (100), and is inserted into a patient's tissue to obtain tissue samples. These tissue samples are communicated through needle and into a tissue handling assembly (300) with a tissue transport tube (302) connected at the proximal end of probe (100), as will also be described in greater detail below. It should also be understood that the use of the term “holster” herein should not be read as requiring any portion of probe (100) to be inserted into any portion of holster (200). For instance, in the present example, holster (200) includes a set of prongs (not shown) that are received by at least a portion of probe (100) to releasably secure probe (100) to holster (200). Probe (100) optionally may include one or more resilient tabs (104) that may be pressed inwardly to disengage the prongs, such that a user may simultaneously depress both tabs (104) then pull probe (100) rearwardly and away from holster (200) to decouple probe (100) from holster (200). Of course, a variety of other types of structures, components, features, etc. (e.g., bayonet mounts, latches, clamps, clips, snap fittings, etc.) may be used to provide removable coupling of probe (100) and holster (200). Furthermore, in some biopsy devices (10), probe (100) and holster (200) may be of unitary or integral construction, such that the two components cannot be separated. By way of example only, in versions where probe (100) and holster (200) are provided as separable components, probe (100) may be provided as a disposable component, while holster (200) may be provided as a reusable component. Still other suitable structural and functional relationships between probe (100) and holster (200) will be apparent to those of ordinary skill in the art in view of the teachings herein.

Biopsy device (10) of the present example is configured to mount to a table or fixture, and be used under stereotactic guidance. Of course, biopsy device (10) may instead be used under ultrasound guidance, MRI guidance, PEM guidance, BSGI guidance, or otherwise. It should also be understood that biopsy device (10) may be sized and configured such that biopsy device (10) may be operated by a single hand of a user. In particular, a user may grasp biopsy device (10), insert needle (110) into a patient's breast, and collect one or a plurality of tissue samples from within the patient's breast, all with just using a single hand. Alternatively, a user may grasp biopsy device (10) with more than one hand and/or with any desired assistance. In some settings, the user may capture a plurality of tissue samples with just a single insertion of needle (110) into the patient's breast. Such tissue samples may be pneumatically deposited within at least a portion of tissue handling assembly (300), and later retrieved from tissue handling assembly (300) for further analysis. While examples described herein often refer to the acquisition of biopsy samples from a patient's breast, it should be understood that biopsy device (10) may be used in a variety of other procedures for a variety of other purposes and in a variety of other parts of a patient's anatomy (e.g., prostate, thyroid, etc.). Various exemplary components, features, configurations, and operabilities of biopsy device (10) will be described in greater detail below; while other suitable components, features, configurations, and operabilities will be apparent to those of ordinary skill in the art in view of the teachings herein.

Holster (200) of the present example includes an outer housing (202) that encloses various components that are used to drive various components of probe (100) for the collection of tissue samples. Although not shown, it should be understood that holster (200) of the present example includes one or more gears (not shown) that mesh with corresponding gears of probe (100). In particular, these gears are exposed through an upper portion of outer housing (202) to mesh with corresponding gears of probe (100) when probe (100) and holster (200) are coupled together. This configuration permits holster (200) to communicate rotary motion to probe (100), thereby driving various components of probe (100) for the collection of tissue samples. For instance, the gears may drive an actuation assembly associated with a hollow tubular cutter (150) (see FIG. 1B) within needle (110) to sever tissue samples received within a lateral aperture (114) defined by needle (110). Likewise, other gears may be employed to rotate needle (110).

As noted above, in some examples a gear associated with holster (200) can provide rotation of needle (110) relative to probe (100). In the present example, this rotation is manually initiated by rotating knob (210). In particular, knob (210) is coupled with the gear associated with rotation of needle (110) by a series of gears (not shown) and shafts (not shown), such that rotation of knob (210) rotates needle (110). By way of example only, such a needle rotation mechanism may be constructed in accordance with the teachings of U.S. Pub. No. 2008/0214955, the disclosure of which is incorporated by reference herein. As another merely illustrative example, a needle rotation mechanism may be constructed in accordance with the teachings of U.S. Pub. No. 2010/0160819, the disclosure of which is incorporated by reference herein. In some other versions, needle (110) is rotated by a motor. In still other versions, needle (110) is simply rotated by rotating thumbwheel (116). Various other suitable ways in which rotation of needle (110) may be provided will be apparent to those of ordinary skill in the art in view of the teachings herein. It should also be understood that some versions may provide no rotation of needle (110).

Holster (200) also includes a firing rod (226) and fork (222), which couple with needle (110) and fire needle (110) distally. By way of example only, such firing may be useful in instances where biopsy device (10) is mounted to a stereotactic table fixture or other fixture, with tip (112) adjacent to a patient's breast, such that the needle firing mechanism may be activated to drive needle (110) into the patient's breast. The needle firing mechanism may be configured to drive needle (110) along any suitable range of motion, to drive tip (112) to any suitable distance relative to fixed components of probe (100).

In the present example, the needle firing mechanism is coupled with needle (110) via a firing rod (226) and a firing fork (222). Firing rod (226) and firing fork (222) are unitarily secured together. Firing fork (222) includes a pair of prongs that receive hub member (120) of needle (110) therebetween. The prongs of firing fork (222) are positioned between an annular flange and a thumbwheel of hub member (120), such that needle (110) will translate unitarily with firing rod (226) and fork (222). The prongs nevertheless removably receive hub member (120), such that fork (222) may be readily secured to hub member (120) when probe (100) is coupled with holster (200); and such that hub member (120) may be readily removed from fork (222) when probe (100) is decoupled from holster (200). The prongs are also configured to permit hub member (120) to rotate between the prongs. Other suitable components, configurations, and relationships will be apparent to those of ordinary skill in the art in view of the teachings herein. The internal components of the needle firing mechanism of the present example are configured and arranged as described in U.S. Pat. No. 8,858,465, entitled “Biopsy Device with Motorized Needle Firing,” issued on Oct. 14, 2014, the disclosure of which is incorporated by reference herein.

Holster (200) of the present example is powered by one or more motors (not shown) contained within outer housing (202). These motors are generally configured to drive one or more gears to thereby rotate and translate a tubular cutter (150) disposed within needle (110). Holster (200) also includes a motor (not shown) that is operable to drive firing rod (226), to thereby arm and fire needle (110). All motors referred to herein are contained within holster (200) in the present example and receive power from vacuum control module (400) via cable (90). In addition, data may be communicated between vacuum control module (400) and holster (200) via cable (90). In some other versions, one or more motors are powered by one or more batteries located within holster (200) and/or probe (100). It should therefore be understood that, as with other components described herein, cable (90) is merely optional. As yet another merely illustrative variation, motors may be powered pneumatically, such that cable (90) may be substituted with a conduit communicating a pressurized fluid medium to holster (200). As still other merely illustrative variation, cable (90) may include one or more rotary drive cables that are driven by motors that are located external to holster (200). It should also be understood that two or three of the motors may be combined as a single motor. Other suitable ways in which various the motors may be driven will be apparent to those of ordinary skill in the art in view of the teachings herein.

Probe (100) of the present example includes a needle (110) extending distally from probe (100) that is inserted into a patient's tissue to obtain tissue samples. Such tissue samples are transported proximally through needle (110) and into transport tube (302), where the tissue samples may be deposited within at least a portion of tissue handling assembly (300), as will be described in greater detail below. Vacuum control module (400) is coupled with probe (100) via a valve assembly (500) and tubes (20, 30), which is operable to selectively provide vacuum, saline, atmospheric air, and venting to probe (100). Although the present example is shown with a pair of tubes (20, 30), it should be understood that in some examples one tube may be omitted in lieu of a tissue transport tube (302) discussed below. The internal components of the valve assembly of the present example are configured and arranged as described in U.S. Pub. No. 2013/0218047, entitled “Biopsy Device Valve Assembly,” published Aug. 22, 2013, the disclosure of which is incorporated by reference herein.

As described above, probe (100) may include one or more gears to mesh with corresponding gears of holster (200). These gears are operable to drive a cutter actuation mechanism in probe (100). Probe (100) may also include another gear that is configured to mesh with a corresponding gear of holster (200) to thereby rotate needle (110).

Needle (110) of the present example comprises a cannula (113) having a tissue piercing tip (112), a lateral aperture (114) located proximal to tip (112), and a hub member (120). Tissue piercing tip (112) is configured to pierce and penetrate tissue, without requiring a high amount of force, and without requiring an opening to be pre-formed in the tissue prior to insertion of tip (112). Alternatively, tip (112) may be blunt (e.g., rounded, flat, etc.) if desired. By way of example only, tip (112) may be configured in accordance with any of the teachings in U.S. Pat. No. 8,801,742, entitled “Needle Assembly and Blade Assembly for Biopsy Device,” issued on Aug. 12, 2014, the disclosure of which is incorporated by reference herein. As another merely illustrative example, tip (112) may be configured in accordance with at least some of the teachings in U.S. Pat. No. 9,486,186, entitled “Biopsy Device with Slide-In Probe,” issued on Nov. 8, 2016, the disclosure of which is incorporated by reference herein. Other suitable configurations that may be used for tip (112) will be apparent to those of ordinary skill in the art in view of the teachings herein.

Lateral aperture (114) is sized to receive prolapsed tissue during operation of device (10). Although not shown, it should be understood that a hollow tubular cutter (150) having a sharp distal edge is located within needle (110). Cutter (150) is operable to rotate and translate relative to needle (110) and past lateral aperture (114) to sever a tissue sample from tissue protruding through lateral aperture (114). For instance, cutter (150) may be moved from an extended position to a retracted position, thereby “opening” lateral aperture (114) to allow tissue to protrude therethrough; then from the retracted position back to the extended position to sever the protruding tissue. As will be described in greater detail below, needle (110) may be rotated to orient lateral aperture (114) at any desired angular position about the longitudinal axis of needle (110). Such rotation of needle (110) is facilitated in the present example by hub member (120), which is described in greater detail below.

In some examples, needle (110) also includes a longitudinal wall (not shown) extending proximally from the proximal portion of tip (112). In such examples, the wall may only extend along a length less than the full length of cannula (113). However, in other examples such a wall may extend the full length of cannula (113) if desired. Where needle (110) includes the wall, the wall may define a two-lumen configuration within needle (110). In examples where the wall only extends for a portion of needle (110), it should be understood that at least a portion of cutter (150) may also define the two-lumen configuration of needle (110). Furthermore, to facilitate fluid flow between lumens, the wall may include a plurality of openings (not shown). An example of such a configuration is disclosed in U.S. Pat. No. 7,918,803, entitled “Methods and Devices for Automated Biopsy and Collection of Soft Tissue,” issued Apr. 5, 2011, the disclosure of which is incorporated by reference herein. Of course, as with any other component described herein, any other suitable configurations may be used.

Hub member (120) of the present example is overmolded about needle (110), such that hub member (120) and needle (110) rotate and translate unitarily with each other. By way of example only, needle (110) may be formed of metal, and hub member (120) may be formed of a plastic material that is overmolded about needle (110) to unitarily secure and form hub member (120) to needle (110). Hub member (120) and needle (110) may alternatively be formed of any other suitable material(s), and may be secured together in any other suitable fashion. In the present example, hub member (120) defines a thumbwheel feature to provide manual rotation of needle. Various other suitable ways in which manual rotation of needle (110) may be provided will be apparent to those of ordinary skill in the art in view of the teachings herein. It should also be understood that rotation of needle (110) may be automated in various ways, including but not limited to the various forms of automatic needle rotation described in various references that are cited herein.

As noted above, needle (110) contains a hollow tubular cutter that is operable to simultaneously translate and rotate relative to needle (110) to sever a tissue sample from tissue protruding through lateral aperture (114). Although not shown, it should be understood that in some examples such a cutter (150) may be operatively coupled to a cutter drive mechanism disposed within probe. Such a cutter drive mechanism may be in communication with one or more gears, which may mesh with one or more corresponding gears of holster (200). The cutter drive mechanism may thus be driven by gears of holster (200) to simultaneously rotate and translate cutter (150) disposed within needle (110). In some examples, cutter (150) drive mechanism may include various threaded and keyed features to facilitate simultaneous rotation and translation of cutter (150). In such examples, a single rotatory input may be converted by the cutter drive mechanism into both rotation and translation of cutter (150). Alternatively, in other examples, rotation and translation of cutter (150) may be supplied by separate rotary inputs. In yet other examples, both rotation and translation of cutter (150) may be provided by two rotary inputs acting together such as by two gears moving at different rotational speeds. In some versions, the foregoing cutter actuation components are further configured in accordance with at least some of the teachings of U.S. Pat. No. 9,345,457, entitled “Presentation of Biopsy Sample by Biopsy Device,” issued on May 24, 2016, the disclosure of which is incorporated by reference herein. As yet another merely illustrative example, cutter (150) disposed within needle (110) may be rotated and/or translated using pneumatic motors, etc. Still other suitable ways in which cutter (150) disposed within needle (110) may be actuated will be apparent to those of ordinary skill in the art in view of the teachings herein.

### II. Exemplary Tissue Handling Assembly

Tissue handling assembly (300) is best seen in FIGS. 1-4. As seen in FIG. 1, at least a portion of tissue handling assembly (300) is incorporated into vacuum control module (400). However, it should be understood that in other examples, tissue handling assembly (300) may be entirely independent and separate from vacuum control module (400). Tissue handling assembly (300) of the present example includes a tissue transport tube (302), and a tissue handler (310). Tissue transport tube (302) extends from tissue handler (310) to biopsy device (10). As will be described in greater detail below, tissue transport tube (302) is generally configured to receive tissue samples from cutter (150) disposed within needle (110) and to communicate such tissue samples from biopsy device (10) to tissue handler (310).

Tissue handler (310) generally includes an outer housing (312) and a collection drawer (320). As will be described in greater detail below, tissue handler (310) is generally configured to receive a plurality of tissue samples from tissue transport tube (302). The tissue samples are then generally arranged in a predetermined configuration. As will also be described in greater detail below, tissue hander (310) may include various sample analysis features to provide procedure room analysis of tissue samples collected within tissue handler (310). Suitable analysis features may include, among other things, x-ray transmitters and receivers, CCD cameras for visual inspection, bioimpedance sensors, and/or etc.

Collection drawer (320) is received within a portion of outer housing (312). Collection drawer (320) is generally translatable into and out of outer housing (312) to provide access to the interior of collection drawer (320) and thereby permit removal of tissue samples and/or various components from tissue handler (310), as will be described in greater detail below. In some examples, collection drawer (320) is manually operable to translate relative to outer housing (312). In other examples, translation of collection drawer (310) is powered by motor driven or pneumatically driven assemblies to provide automatic or semi-automatic translation of collection drawer (310).

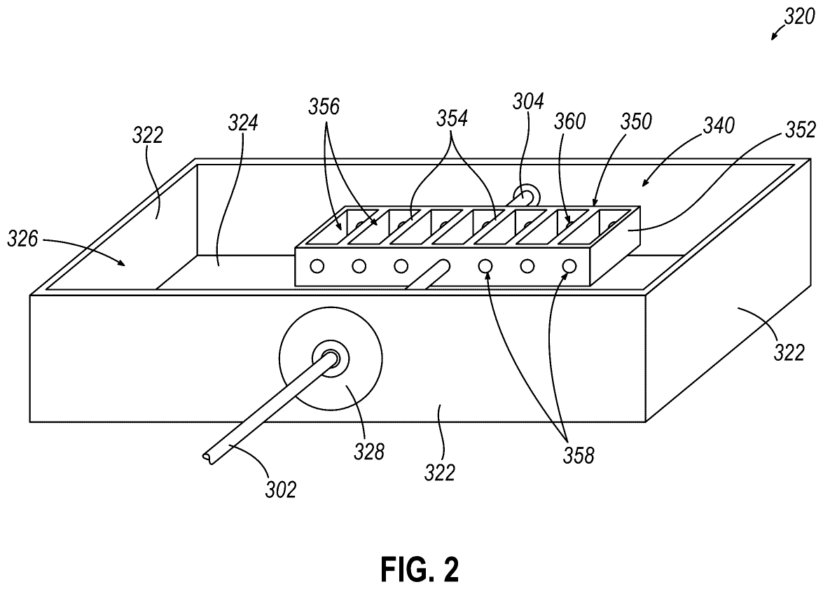

As best seen in FIG. 2, the interior of collection drawer (320) includes a plurality of sidewalls (322) and a floor (324) defining an interior space (326). Tissue transport tube (302) is configured to communicate with the interior space (326) of collection drawer (320) through a tube port (328) disposed in at least one sidewall (322) of collection drawer (320). Although not shown, it should be understood that in some examples, sidewalls (322) and/or floor (324) may include one or more ports to provide draining of fluid from collection drawer (320). For instance, in some instances biopsy device (10) is used with saline or other fluid mediums. Through the tissue sample collection process, an at least some refuse fluids from the biopsy procedure may flow into interior space (326) of collection drawer (320). Thus, it may be desirable in such examples to include drainage ports through sidewalls (322) and/or floor (324).

In the present example, collection drawer (320) contains a tissue sample holder (340) within interior space (326) defined by sidewalls (322) and floor (324). Tissue sample holder (340) of the present example is generally in communication with tissue transport tube (302) to receive a plurality of tissue samples in a predetermined arrangement or configuration. Tissue sample holder (340) of the present example incudes a sample tray (350) having an elongate rectangular structure and configured to translate within collection drawer (320) relative to tissue transport tube (302). As will be described in greater detail below, sample tray (350) is generally of a cassette-like configuration such that sample tray (350) is translatable within collection drawer (320) to successively collect tissue samples within discrete portions of sample tray (350). In some examples, tissue sample holder (340) may further include one or more tray containers, covers, or casings, which may be configured to removably receive sample tray (350). In such examples, such tray containers may be desirable to control vacuum applied to sample tray (350) or to manage fluid communicated into one or more portions of sample tray (350). Of course, in other versions, such tray containers are entirely optional and may be omitted.

Sample tray (350) of the present example includes a rectangular body (352) with a plurality of divider walls (354) defining a plurality of sample chambers (356). A front portion of rectangular body (352) defines a plurality of receiving openings (358) in communication with each respective sample chamber (356). Meanwhile, a rear portion of rectangular body (352) defines a plurality of vacuum openings (360), also in communication with each respective sample chamber (356). Each receiving opening (358) is configured to independently communicate with tissue transport tube (302) so that one or more tissue samples may be communicated into a respective sample chamber (356). Correspondingly, each vacuum opening (360) is configured to communicate with a vacuum tube (304) disposed opposite of tissue transport tube (302) such that vacuum may be communicated into a respective sample chamber (356) to draw one or more tissue samples into the respective sample chamber (356) under vacuum communicated via vacuum tube (304).

An upper portion of rectangular body (352) is generally open or otherwise exposed to the exterior of sample tray (350) such that each sample chamber (356) may be accessible from the upper portion of rectangular body (352). In other examples, one or more portions of rectangular body (352) may be configured to be selectively covered such as by a door, cover, casing, and/or etc. Such selective covering may be desirable in some examples to control the flow of vacuum through sample chambers (356), particularly during transport of one or more tissue samples. Thus, in in some examples, each sample chamber (356) may be independently coverable so only the particular sample chamber (356) receiving one or more tissue samples may be covered. In other examples, all sample chambers (356) may be covered simultaneously. In still other examples, such covering may be omitted and vacuum may be controlled by other components such as fixtures or housings associated with collection drawer (320).

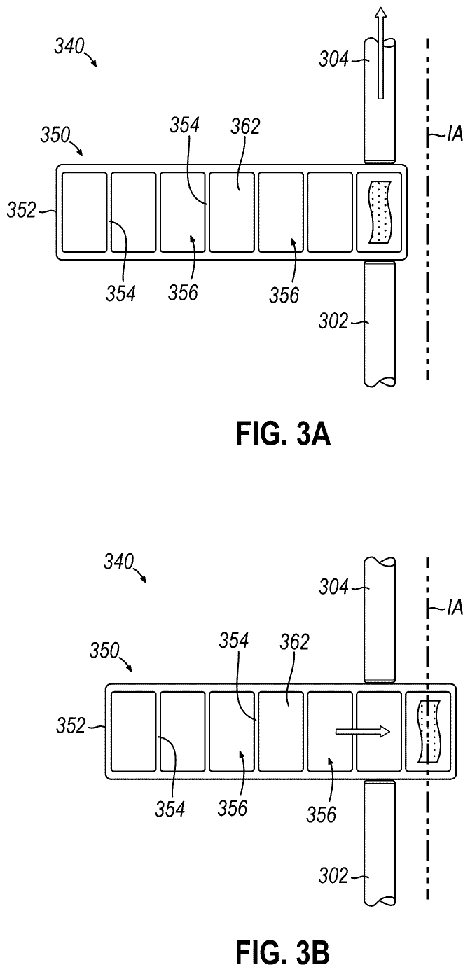

A lower portion of rectangular body (352) includes a floor (362) (see FIGS. 3A and 3B). In the present example, floor (362) is formed of a generally solid material. While floor (362) of the present example is generally non-permeable, floor (362) may have at least some permeability in some examples. For instance, in some examples floor (362) includes a semi-permeable membrane or a mesh configured to permit fluids to pass through floor (362). Such permeability may be desirable to control fluid entering sample tray (350). Thus, in other examples, floor (362) is generally non-permeable, but may include other fluid management features such as ports, valves, and/or etc. In still other examples, fluid may be controlled through vacuum tube (304).

As best seen in FIG. 3A, sample tray (350) is disposed between a proximal end of tissue transport tube (302) and a distal end of vacuum tube (304) with transport tube (302) and vacuum tube (304) aligned along a common axis extending generally perpendicular relative to the longitudinal axis of sample tray (350). Thus, tissue transport tube (302) and vacuum tube (304) are each configured to simultaneously be in communication with a selected sample chamber (356) of the plurality of sample chambers (356) depending on the axial position of sample tray (350) relative to tissue transport tube (302) and vacuum tube (304). To facilitate such communication, one or more portions of sample tray (305) may include slidable seals, guides, or other structures configured to permit sealing of tissue transport tube (302) and vacuum tube (304) with each opening (358, 360), while permitting sliding of sample tray (350) relative to tissue transport tube (302) and vacuum tube (304).

As described above, sample tray (350) is generally configured to translate relative to tissue transport tube (302) and vacuum tube (304). In some examples, tissue sample holder (340) includes one or more translation mechanisms configured to facilitate translation of sample tray (350). It should be understood such translation mechanisms may take on a variety of forms such as linear actuators, rack-based translators, wheel-based translators, and/or etc. Such mechanisms may additionally be motor driven or mechanically driven (e.g., by spring-loaded mechanisms). In yet other examples, translation of sample tray (350) may be controlled manually by an operator. In still other examples, sample tray (350) is fixed within collection drawer (320), while transport tube (302) and vacuum tube (304) are configured to translate by one or more translation mechanisms similar to those described above.

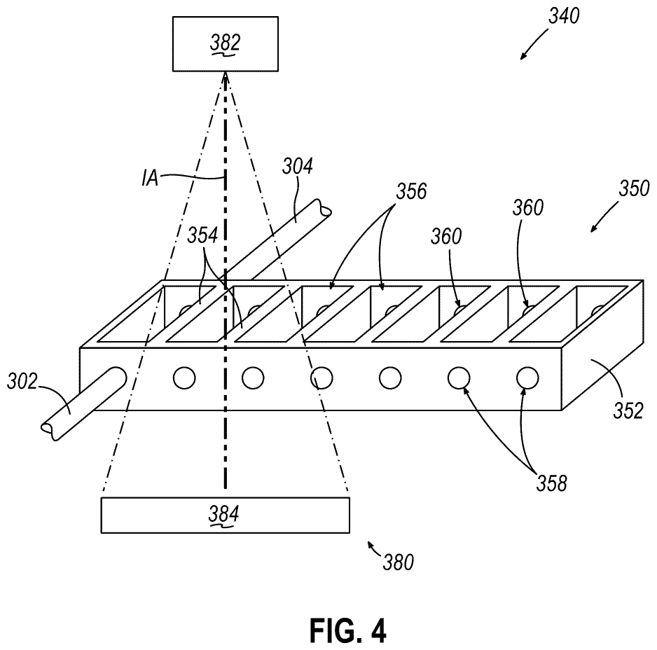

As best seen in FIGS. 3A, 3B and 4, in use, sample tray (350) is configured to receive tissue samples in each sample chamber (356) and image each received tissue sample upon receipt. As can be seen in FIG. 3A, sample tray (350) may be initially aligned relative to tissue transport tube (302) and vacuum tube (304) so that a predetermined initial sample chamber (356) is aligned with tissue transport tube (302) and vacuum tube (304). In the present example, the predetermined initial sample chamber (356) corresponds to the right-most sample chamber (356), although any other sample chamber (356) may be used in other examples.

Regardless of the particular predetermined initial sample chamber (356), a tissue sample may be communicated into the predetermined initial sample chamber (356) via tissue transport tube (302) and vacuum tube (304) operating cooperatively to transport the tissue sample from biopsy device (10) and into the predetermined initial sample chamber (356) using vacuum supplied by vacuum tube (304). Although not shown, it should be understood sample tray (350) and/or vacuum tube (304) can include one or more tissue stopping structures configured to prevent the tissue sample from progressing out of a given sample chamber (356) and into vacuum tube (304).

Once the tissue sample is received within the predetermined initial sample chamber (356) as shown in FIG. 3A, sample tray (350) may be advanced to position the predetermined initial sample chamber (356) into alignment with an imaging axis (IA) as can be seen in FIG. 3B. In the present example, this advancement is show as progressing sample tray (350) to the right by a single sample chamber (356). However, it should be understood that different advancement profiles or patterns may be used in other examples. In the present example, the single sample chamber (356) advancement profile may be desirable to also simultaneously align the next adjacent open sample chamber (356) with tissue transport tube (302) and vacuum tube (304) for communication of another tissue sample simultaneously with imaging.

Once sample tray (350) is advanced as shown in FIG. 3B, the tissue sample may disposed within the predetermined initial sample chamber (356) is imaged. In particular, as best seen in FIG. 4, tissue handling assembly (300) further includes an analysis assembly (380) aligned with the imaging axis (IA). Analysis assembly (380) is generally configured to analyze one or more properties of tissue. For instance, in the present example, analysis assembly (380) includes an x-ray source (382) and an x-ray detector (384). Both x-ray source (382) and x-ray detector (384) are operable to cooperatively image a tissue sample when the tissue sample is aligned with the imaging axis (IA). Such x-ray based-imaging may be desirable in some examples to detect the presence of one or more calcifications within the tissue sample in real-time, thereby providing feedback to an operator that may be used for the collection of subsequent tissue samples.

After analysis is performed using analysis assembly (380), another tissue sample may be collected within another sample chamber (356). As described above, subsequent collection of another tissue sample may also occur concurrently with analysis or imaging in some examples. Regardless, the process of tissue sample collection and analysis or imaging may then be repeated to successively fill each sample chamber (356) of sample tray (350) or until a desired number of tissue samples has been collected.

### III. Exemplary Alternative Sample Trays

As described above, tissue sample holder (340) includes sample tray (350) with a generally cassette-like configuration. However, in some examples, it may be desirable to incorporate different sample trays having different configurations into tissue sample holder (340). Such alternative sample tray configurations may be desirable to provide the real-time sample analysis functionality described above in combination with certain alternative functionalities to achieve different operational objectives. For instance, in some circumstances different dimensions or operational footprints may be desirable. In addition, or in the alternative, different functionalities may be desirable such as separating aspects of sample analysis from sample storage. Although certain specific alternative sample trays (450, 550) are described below, it should be understood that such alternative sample trays (450, 550) may include one or more features of sample tray (350) described above. Additionally, in some examples, one or more of sample trays (350, 450, 550) describe herein may be used in combination with other sample trays (350, 450, 550).

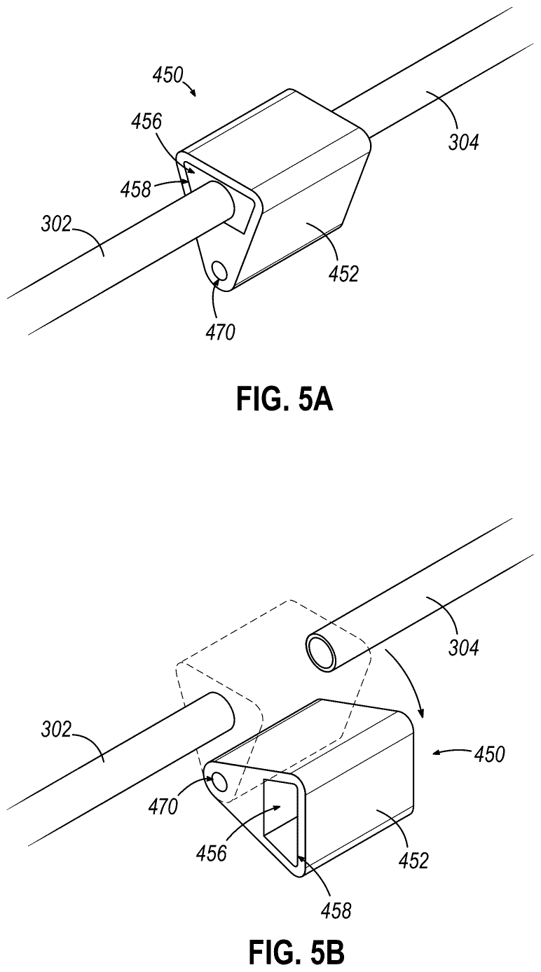

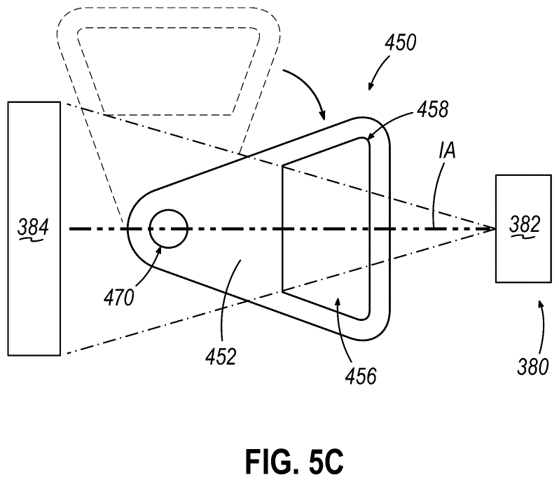

FIGS. 5A and 5B show an exemplary alternative sample tray (450) that may be readily incorporated into tissue sample holder (350) described above. Sample tray (450) is generally configured to receive one or more tissue samples within a portion thereof and manipulate such received tissue samples for further analysis or imaging. As will be understood, sample tray (450) of the present example may be used either in combination with sample tray (350) or in-lieu of sample tray (350).

As best seen in FIG. 5A, sample tray (450) of the present example includes a body (452) defining a sample chamber (456) and a pivot (470). Body (452) generally defines prism-shaped or pie-shaped exterior with sample chamber (456) oriented proximate the wider portion of body (452) and pivot (470) oriented proximate the narrower portion of body (452). As will be described in greater detail below, body (452) is generally configured to move relative to tissue transport tube (302) and/or vacuum tube (304) to move sample chamber (456) between a tissue transport axis defined by tissue transport tube (302) and an imaging axis (IA).

Sample chamber (456) generally extends longitudinally though body (452) from a distal end of body (452) to a proximal end of body (452). Thus, sample chamber (456) defines an open distal end (458) and an open proximal end (not shown). Open distal end (458) is configured to selectively communicate with tissue transport tube (302), while the open proximal end is configured to selectively communicate with vacuum tube (304). As will be described in greater detail below, sample chamber (456) is configured to receive one or more tissue samples from tissue transport tube (302) through open distal end (458). Optionally, one or more of the received tissue samples may transported from sample chamber (456) via the open proximal end and vacuum tube (304). Thus, in some examples, sample tray (450) is configured as a temporary tissue sample storage container where one or more tissue samples may be held for imaging or analysis purposes before being transported to another container for ultimate tissue sample storage.

Sample chamber (456) generally defines a rectangular or trapezoidal cross-sectional shape, although other shapes may be used. In the present example, the shape of sample chamber (456) is generally a function of the shape of body (452). Thus, the shape of sample chamber (456) generally corresponds to the shape of the wider portion of body (452). In other examples, sample chamber (456) has a shape that is independent of the shape of body (452). For instance, in some examples, sample chamber (456) is cylindrical or oval-shaped. Of course, various alternative shapes for sample chamber (456) may be used as will be apparent to those of ordinary skill in the art in view of the teachings herein.

Body (452) of the present example defines a single sample chamber (456) generally sized to receive a single tissue sample. In other examples, the particular configuration of sample chamber (456) may be varied. For instance, in some examples, body (452) defines a plurality of sample chambers (456). In addition, or in the alternative, body (452) defines differently sized sample chambers (456) configured for receipt of a plurality of tissue samples.

As described above, sample chamber (456) is configured to communicate with tissue transport tube (302) and/or vacuum tube (304). Thus, it should be understood that body (452) and/or sample chamber (456) can be equipped with certain sealing features configured to engage tissue transport tube (302) and/or vacuum tube (304). By way of example only, suitable sealing features may include o-rings and/or gaskets in combination with other structural features such as housings, fixtures, springs, and/or etc. Such sealing features are generally configured to promote sealing between sample chamber (456) and tubes (302, 304), while permitting selective movement of body (452) relative to tubes (302, 304).

Pivot (470) is positioned proximate the narrower portion of body (452) or opposite the position of sample chamber (456). Pivot (470) generally defines an axis about which body (452) can pivot. Although not shown, it should be understood that pivot (470) may include other structural features such as rods, bearings, blocks, pivot points, levers, arms, and/or etc. For instance, in some examples, pivot (470) is connected to a rod, which is in communication with a motor to drive rotation of pivot (470) via the rod.

As best seen in FIGS. 5A through 5C, sample tray (450) is used to collect a tissue sample from tissue transport tube (302) along a sampling axis and then move away from the sampling axis in-line with an imaging axis to image or otherwise analyze the collected tissue sample. As best seen in FIG. 5A, sample tray (450) is initially positioned to align sample chamber (456) with a sampling axis defined by tissue transport tube (302) and/or vacuum tube (304). In this alignment, vacuum is supplied to the open proximal end of sample tray (450) to draw a tissue sample though tissue transport tube (302) and into sample chamber (456) through open distal end (458). Optionally, in some examples, positive pressure saline is used to push the tissue sample into sample chamber (456) in addition to or in-lieu of vacuum from vacuum tube (304).

Once the tissue sample is received within sample chamber (456), sample tray (450) includes one or more features to stop the progression of the tissue sample, thereby holding the tissue sample within sample chamber (456). Suitable structures to stop the progression of the tissue sample may include, for example, movable or fixed screens or meshes, movable gates, flexible seals, one or more tapered features with sample chamber (456), and/or etc. In other examples, stopping the progression of the tissue sample may be facilitated functionally. For instance, the source of vacuum and/or saline may be closed to eliminate force applied to the tissue sample for movement along the sampling axis. In such examples, closing of the source of vacuum and/or saline may be based on a count or timer. In other examples, closing of the source of vacuum and/or saline may be based on a sensor associated with sample tray (450), which may be used to detect the presence of the tissue sample within sample chamber (456).

Once the progression of the tissue sample has ceased within sample chamber (456), it may be desirable to image or otherwise analyze the tissue sample received within sample chamber (456). Such imaging or other analysis may be desirable to provide real-time feedback to inform an operator for the collection of additional tissue samples. For instance, if one or more calcifications are detected in the tissue sample, an operator may collect additional samples within a similar area. Alternatively, if no calcifications are detected in the tissue sample, an operator may reposition biopsy device (10) to collect additional tissue samples from another area.

As best seen in FIGS. 5B and 5C, analysis of the tissue sample received within sample chamber (456) is performed by rotating or moving sample tray (450) about pivot (470) and relative to tubes (302, 304). This moves sample chamber (456) away from the sampling axis and into alignment with imaging axis (IA) defined by x-ray source (382) and x-ray detector (384) of analysis assembly (380). Once sample chamber (456) is moved into alignment with imaging axis (IA), x-ray source (382) and x-ray detector (384) are activated to image the tissue sample.

After imaging and/or analysis of the tissue sample, it may be desirable to remove the tissue sample from sample chamber (456) to clear sample chamber (456) for the collection of one or more additional tissue samples. For instance, in the present example, sample tray (450) is moved back into alignment with the sampling axis (see FIG. 5A). Once sample tray (450) moved back into alignment with the sampling axis, vacuum tube (304) may be used to transport the tissue sample from sample chamber (456) to one or more additional sample trays of tissue sample holder (340) configured to hold tissue samples. It should be understood such additional sample trays may take on a variety of configurations such as cassette-style configurations, rotary-style configurations, or bulk-style configurations (e.g., single chamber sample basket). Although the present example uses vacuum tube (304) for post-imaging or analysis transport of the tissue sample, it should be understood that in other examples, a dedicated transport tube may be used. For instance, in some examples, another transport tube may be aligned with imaging axis (IA). After imaging and/or analysis, the other transport tube may be used to transport the tissue sample without requiring movement of sample tray (450). In still other examples, subsequent removal of the tissue sample may be manually-based with an operator manually removing the tissue sample from sample chamber (456).

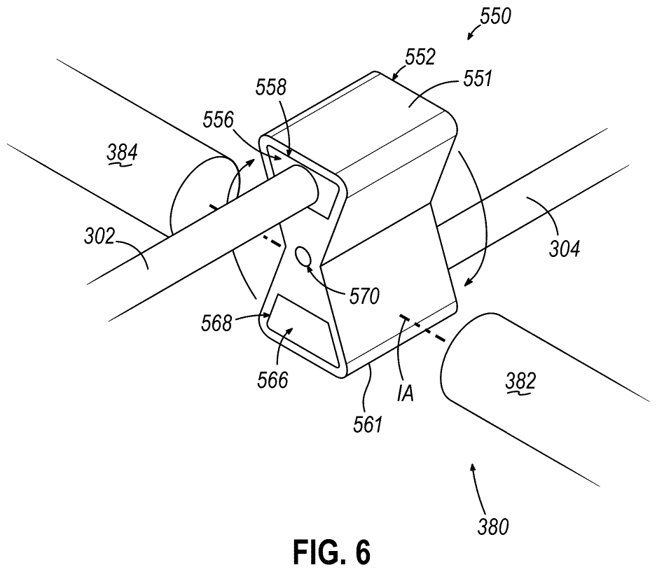

FIG. 6 shows an exemplary alternative sample tray (550) that is similar to sample tray (450) described above. For instance, like with sample tray (450), sample tray (550) of the present example is generally configured to receive one or more tissue samples within a portion thereof and manipulate such received tissue samples for further analysis or imaging. As will be understood, sample tray (550) of the present example may be used either in combination with sample trays (350, 450) described above or in-lieu of sample trays (350, 450).

As can be seen, sample tray (550) of the present example includes a body (552) defining a first portion (551), a second portion (561), and a pivot (570). Each of first portion (551) and second portion (561) define a respective sample chamber (556, 566) with pivot (470) disposed between each sample chamber (556, 566). Thus, unlike body (452) described above, body (552) of the present example generally defines double prism-shaped or double pie-shaped exterior with sample chambers (556, 566) oriented proximate each the wider portion of body (552) and pivot (570) oriented proximate the narrower portion of body (552). In other words, body (552) defines a generally hour glass-shaped cross-section with two wider portions opposite a narrower portion. As will be described in greater detail below, body (552) is generally configured to move relative to tissue transport tube (302) and/or vacuum tube (304) to move sample chambers (556, 566) between a tissue transport axis defined by tissue transport tube (302) and an imaging axis (IA).

Each sample chamber (556, 566) generally extends longitudinally though body (552) from a distal end of body (552) to a proximal end of body (552). Thus, each sample chamber (556, 566) defines a respective open distal end (558, 568) and an open proximal end (not shown). Each open distal end (558, 568) is configured to selectively communicate with tissue transport tube (302), while each respective open proximal end is configured to selectively communicate with vacuum tube (304). As will be described in greater detail below, each sample chamber (556, 566) is configured to receive one or more tissue samples from tissue transport tube (302) through its respective open distal end (558, 568). Optionally, one or more of the received tissue samples may transported from each sample chamber (556, 566) via the open proximal end and vacuum tube (304). Thus, in some examples, sample tray (550) is configured as a temporary tissue sample storage container where one or more tissue samples may be held for imaging or analysis purposes before being transported to another container for ultimate tissue sample storage.

Each sample chamber (556, 566) generally defines a rectangular or trapezoidal cross-sectional shape, although other shapes may be used. In the present example, the shape of each sample chamber (556, 566) is generally a function of the shape of body (552). Thus, the shape of each sample chamber (556, 566) generally corresponds to the shape of the wider portions of body (552). In other examples, each sample chamber (556, 566) has a shape that is independent of the shape of body (552). For instance, in some examples, each sample chamber (556, 566) is cylindrical or oval-shaped. In addition, or in the alternative, each sample chamber (556, 566) defines a different shape relative to the other sample chambers (566, 556). Of course, various alternative shapes for each sample chamber (556, 566) may be used as will be apparent to those of ordinary skill in the art in view of the teachings herein.

Body (552) of the present example defines a single sample chamber (556, 566) with respect to each portion (551, 561). Additionally, each chamber (556, 566) is generally sized to receive a single tissue sample. In other examples, the particular configuration of each sample chamber (556, 566) may be readily varied, either independently of each other or identically. For instance, in some examples, body (552) defines a plurality of sample chambers (556, 566) within each portion (551, 561). In addition, or in the alternative, body (552) defines differently sized sample chambers (556, 566) configured for receipt of a plurality of tissue samples.

As described above, each sample chamber (556, 566) is configured to communicate with tissue transport tube (302) and/or vacuum tube (304). Thus, it should be understood that body (552) and/or each sample chamber (556, 566) can be equipped with certain sealing features configured to engage tissue transport tube (302) and/or vacuum tube (304). By way of example only, suitable sealing features may include o-rings and/or gaskets in combination with other structural features such as housings, fixtures, springs, and/or etc. Such sealing features are generally configured to promote sealing between each sample chamber (556, 566) and tubes (302, 304), while permitting selective movement of body (552) relative to tubes (302, 304).

In addition to the sealing features described above, body (552) and/or each sample chamber (556, 566) are configured with certain vacuum management features in some examples. For instance, as seen in FIG. 6, vacuum tube (304) is laterally offset relative to tissue transport tube (302) in some examples. As will be described in greater detail below, such a configuration may be desirable to facilitation of acquisition of a tissue sample within one sample chamber (556, 566), while another tissue sample is transported away from another sample chamber (566, 556). In such examples, it may still be desirable to supply vacuum to tissue transport tube (302) via a given sample chamber (556, 566). Thus, such vacuum management features may be configured to communicate vacuum from vacuum tube (304) to one or both of sample chambers (556, 566) either selectively or continuously. By way of example only, suitable vacuum management features include manifolds, one or more passageways extending through body (552), additional tubes, and/or etc. In other examples, vacuum is communicated to each sample chamber (556, 566) separately via a dedicated vacuum tube for each sample chamber (556, 566).

Pivot (570) is positioned proximate the narrower portion of body (552) or between each sample chamber (556, 566). Pivot (570) generally defines an axis about which body (552) can pivot. Although not shown, it should be understood that pivot (570) may include other structural features such as rods, bearings, blocks, pivot points, levers, arms, and/or etc. For instance, in some examples, pivot (570) is connected to a rod, which is in communication with a motor to drive rotation of pivot (570) via the rod.

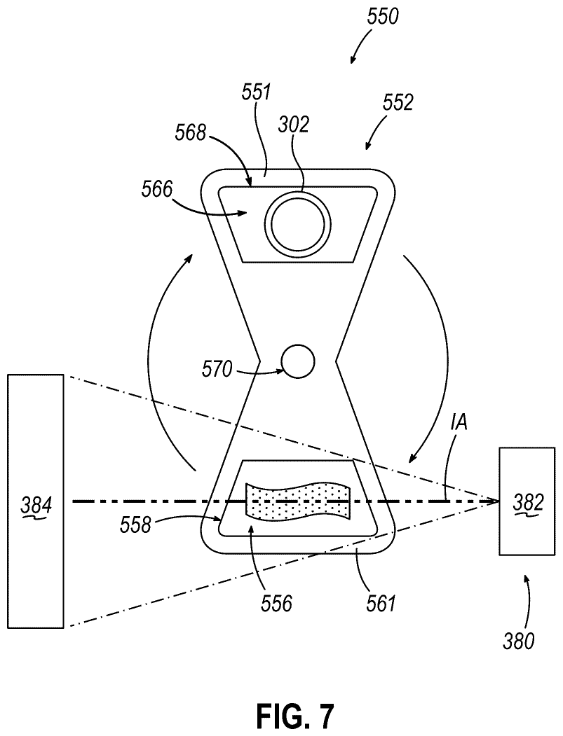

As best seen in FIGS. 6 and 7, sample tray (550) is used to collect a tissue sample from tissue transport tube (302) along a sampling axis and then move away from the sampling axis in-line with an imaging axis to image or otherwise analyze the collected tissue sample. However, unlike sample tray (450) described above, sample tray (550) of the present example is configured to simultaneously collect a tissue sample and image or otherwise analyze a tissue sample using both sample chambers (556, 566). As best seen in FIG. 6, sample tray (550) is initially positioned to align a first sample chamber (556) with a sampling axis defined by tissue transport tube (302). Meanwhile, a second sample chamber (566) is aligned with imaging axis (IA) such that imaging or other analysis may occur with respect to second sample chamber (556), while a tissue sample is collected within first sample chamber (556). In this alignment, vacuum is optionally supplied to the open proximal end of sample tray (550) and may be communicated to first sample chamber (556) via a manifold or other vacuum management feature to draw a tissue sample though tissue transport tube (302) and into first sample chamber (556) through open distal end (558). Optionally, in some examples, positive pressure saline is used to push the tissue sample into first sample chamber (556) in addition to or in-lieu of vacuum from vacuum tube (304).

Once the tissue sample is received within first sample chamber (556), sample tray (550) includes one or more features to stop the progression of the tissue sample, thereby holding the tissue sample within first sample chamber (556). Suitable structures to stop the progression of the tissue sample may include, for example, movable or fixed screens or meshes, movable gates, flexible seals, one or more tapered features with first sample chamber (556), and/or etc. In other examples, stopping the progression of the tissue sample may be facilitated functionally. For instance, the source of vacuum and/or saline may be closed to eliminate force applied to the tissue sample for movement along the sampling axis. In such examples, closing of the source of vacuum and/or saline may be based on a count or timer. In other examples, closing of the source of vacuum and/or saline may be based on a sensor associated with sample tray (550), which may be used to detect the presence of the tissue sample within first sample chamber (556).

Once the progression of the tissue sample has ceased within first sample chamber (556), it may be desirable to image or otherwise analyze the tissue sample received within first sample chamber (556). As best seen in FIGS. 6 and 7, analysis of the tissue sample received within first sample chamber (556) is performed by rotating or moving sample tray (550) about pivot (570) and relative to tubes (302, 304). This moves first sample chamber (556) away from the sampling axis and into alignment with imaging axis (IA) defined by x-ray source (382) and x-ray detector (384) of analysis assembly (380). Simultaneously, this movement moves second sample chamber (566) away from imaging axis (IA) and into alignment with the sampling axis. Once first sample chamber (556) is moved into alignment with imaging axis (IA), x-ray source (382) and x-ray detector (384) are activated to image the tissue sample. Additionally, once second sample chamber (566) is moved into alignment with the sampling axis, second sample chamber (566) is configured to receive another tissue sample as similarly described above with first sample chamber (556). In some examples, such receipt of another tissue sample may optionally occur contemporaneously with imaging or other analysis via analysis assembly (380).

After imaging and/or analysis of the tissue sample, it may be desirable to remove the tissue sample from first sample chamber (556) to clear first sample chamber (556) for the collection of one or more additional tissue samples. For instance, in the present example, vacuum tube (304) is positioned in an alignment with imaging axis (IA). Thus, vacuum tube (304) is used in the present example to remove the tissue sample from first sample chamber (556) after imaging or other analysis without additional movement of body (552). Meanwhile, second sample chamber (566) may receive another tissue sample therein either simultaneously, before, or after. Vacuum tube (304) may be used to transport the tissue sample from first sample chamber (556) to one or more additional sample trays of tissue sample holder (340) configured to hold tissue samples. It should be understood such additional sample trays may take on a variety of configurations such as cassette-style configurations, rotary-style configurations, or bulk-style configurations (e.g., single chamber sample basket). Although the present example uses vacuum tube (304) for post-imaging or analysis transport of the tissue sample, it should be understood that in other examples, a dedicated transport tube may be used. For instance, in some examples, a dedicated transport tube may be aligned with imaging axis (IA), while vacuum tube (304) may be aligned with sampling axis. After imaging and/or analysis, the other transport tube may be used to transport the tissue sample, also without requiring movement of sample tray (450). In still other examples, subsequent removal of the tissue sample may be manually-based with an operator manually removing the tissue sample from first sample chamber (556). After the tissue sample is removed from first sample chamber (556), the same process described above may be repeated for second sample chamber (566).

### IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy system, the biopsy system comprising: a biopsy device, the biopsy device including: a probe, a needle extending from the probe, and a cutter, the cutter being movable relative to the needle to sever one or more tissue samples; a control module in communication with the biopsy device, the control module including a tissue sample holder including a sample tray, the sample tray being configured to receive the one or more tissue samples severed by the cutter; and a tissue transport tube adapted to connect between the tissue sample holder and the cutter of the biopsy device, the sample tray having a sampling configuration and an imaging configuration, the sample tray being configured to move between a sampling axis corresponding to the sampling configuration and an imaging axis corresponding to the imaging configuration to receive and subsequently image one or more tissue samples received within the sample tray.

### Example 2

The biopsy system of Example 1, the sample tray including a rectangular body defining a plurality of sample chambers arranged along a longitudinal axis of the rectangular body.

### Example 3

The biopsy system of Example 2, the sample tray being configured to translate in a direction parallel to the longitudinal axis of the body to move a sample chamber of the plurality of sample chambers from the sampling axis to the imaging axis.

### Example 4

The biopsy system of Examples 2 or 3, the sample tray defining a plurality of receiving openings, each receiving opening corresponding to a sample chamber of the plurality of sample chambers, each receiving opening being configured to selectively communicate with the tissue transport tube.

### Example 5

The biopsy system of Example 4, the sample tray further defining a plurality of vacuum openings, each vacuum opening corresponding to a sample chamber of the plurality of sample chambers, each vacuum opening being configured to selectively communicate with a vacuum tube to thereby induce a vacuum within a given sample chamber of the plurality of sample chambers.

### Example 6

The biopsy system of Example 5, the tissue transport tube and the vacuum tube defining the sampling axis.

### Example 7

The biopsy system of any of Examples 1 through 5, the sample tray being configured to translate relative to a proximal end of the transport tube.

### Example 8

The biopsy system of Example 1, the sample tray including a body defining a pivot and a first sample chamber, the first sample chamber being offset relative to the pivot and configured to receive one or more tissue samples therein.

### Example 9

The biopsy system of Example 8, the body being configured to rotate relative to the pivot to move the first sample chamber between the sapling axis and the imaging axis.

### Example 10

The biopsy system of Examples 8 or 9, the body defining a first portion and a second portion, the first portion being opposite the second portion, the first sample chamber being defined by the first portion, the second portion defining a second sample chamber.

### Example 11

The biopsy system of Example 10, the pivot being positioned between the first portion and the second portion, the body being configured to rotate relative to the pivot to alternatingly move the first sample chamber and the second sample chamber between the sapling axis and the imaging axis.

### Example 12

The biopsy system of Examples 8 or 9, the body defining a triangular cross-sectional shape.

### Example 13

The biopsy system of Examples 8 or 9, the body defining a hourglass cross-sectional shape.

### Example 14

The biopsy system of any of Examples 1 through 13, the tissue transport tube being configured to communicate one or more tissue samples therethrough using vacuum, saline, or a combination thereof.

### Example 15

The biopsy system of any of Examples 1 through 14, further comprising an analysis assembly, the analysis assembly including an x-ray source and an x-ray detector, the x-ray source and the x-ray detector being positioned relative to each other to define the imaging axis.

### Example 16

An apparatus for use with a biopsy device, the apparatus comprising: a tissue transport tube, the tissue transport tube being configured to receive and transport one or more tissue samples acquired by the biopsy device; a tissue sample holder, the tissue sample holder including a tissue tray, the tissue tray being in selective communication with the tissue transport tube to receive the one or more tissue samples acquired by the biopsy device from the tissue transport tube; and an analysis assembly defining an imaging axis extending from a detector, the tissue tray being configured to move relative to the analysis assembly into alignment with the imaging axis to analyze the one or more tissue samples acquired by the biopsy device.

### Example 17

The apparatus of Example 16, the tissue transport tube defining a sampling axis, the sampling axis being offset relative to the imaging axis.

### Example 18

The apparatus of Example 16, further comprising a vacuum tube, the vacuum tube including a distal opening positioned opposite a proximal opening of the tissue transport tube, the vacuum tube being configured to receive one or more tissue samples from the tissue tray.

### Example 19

The apparatus of any of Examples 16 through 18, the analysis including a source and the detector, the source being an x-ray source, the detector being an x-ray detector.

### Example 20

A method of analyzing a tissue sample, the method comprising: transporting a tissue sample acquired by a biopsy device through a tissue transport tube into a sample chamber of a tissue tray aligned with a sampling axis defined by the tissue transport tube; moving the sample chamber of the tissue tray relative to the sampling axis to align the sample chamber with an imaging axis; and imaging the tissue sample while the sample chamber of the tissue tray is aligned with the sampling axis.

### Example 21

The method of Example 20, the step of moving the sample chamber of the tissue tray relative to the sampling axis further including translating the tissue tray to align another sample chamber with the sampling axis.

### Example 22

The method of Example 20, the step of moving the sample chamber of the tissue tray relative to the sampling axis further including rotating the tissue tray relative to a pivot to move the sample chamber relative to the sampling axis.

### Example 23

The method of Example 22, the step of moving the sample chamber of the tissue tray relative to the sampling axis further including moving another sample chamber of the tissue tray into alignment with the sampling axis while moving the sample chamber into alignment with the imaging axis.

### Example 24

The method of Example 20, further comprising moving the tissue tray relative to the sampling axis after imaging the tissue sample to receive another tissue sample within the tissue tray.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Embodiments of the present invention have application in conventional endoscopic and open surgical instrumentation as well as application in robotic-assisted surgery.

By way of example only, embodiments described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta or gamma radiation, ethylene oxide, or steam.

Embodiments of the devices disclosed herein can be reconditioned for reuse after at least one use. Reconditioning may include any combination of the steps of disassembly of the device, followed by cleaning or replacement of particular pieces, and subsequent reassembly. In particular, embodiments of the devices disclosed herein may be disassembled, and any number of the particular pieces or parts of the devices may be selectively replaced or removed in any combination. Upon cleaning and/or replacement of particular parts, embodiments of the devices may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy system, the biopsy system comprising:
(a) a biopsy device, the biopsy device including:
(i) a probe,
(ii) a needle extending from the probe, and
(iii) a cutter, the cutter being movable relative to the needle to sever one or more tissue samples;
(b) a control module in communication with the biopsy device, the control module including a tissue sample holder including a sample tray, the sample tray being configured to receive the one or more tissue samples severed by the cutter; and
(c) a tissue transport tube adapted to connect between the tissue sample holder and the cutter of the biopsy device, the sample tray having a sampling configuration and an imaging configuration, the sample tray being configured to move between a sampling axis corresponding to the sampling configuration and an imaging axis corresponding to the imaging configuration to receive and subsequently image one or more tissue samples received within the sample tray.

2. The biopsy system of claim 1, the sample tray including a rectangular body defining a plurality of sample chambers arranged along a longitudinal axis of the rectangular body.

3. The biopsy system of claim 2, the sample tray being configured to translate in a direction parallel to the longitudinal axis of the body to move a sample chamber of the plurality of sample chambers from the sampling axis to the imaging axis.

4. The biopsy system of claim 2, the sample tray defining a plurality of receiving openings, each receiving opening corresponding to a sample chamber of the plurality of sample chambers, each receiving opening being configured to selectively communicate with the tissue transport tube.

5. The biopsy system of claim 4, the sample tray further defining a plurality of vacuum openings, each vacuum opening corresponding to a sample chamber of the plurality of sample chambers, each vacuum opening being configured to selectively communicate with a vacuum tube to thereby induce a vacuum within a given sample chamber of the plurality of sample chambers.

6. The biopsy system of claim 5, the tissue transport tube and the vacuum tube defining the sampling axis.

7. The biopsy system of claim 1, the sample tray being configured to translate relative to a proximal end of the transport tube.

8. The biopsy system of claim 1, the sample tray including a body defining a pivot and a first sample chamber, the first sample chamber being offset relative to the pivot and configured to receive one or more tissue samples therein.

9. The biopsy system of claim 8, the body being configured to rotate relative to the pivot to move the first sample chamber between the sapling axis and the imaging axis.

10. The biopsy system of claim 8, the body defining a first portion and a second portion, the first portion being opposite the second portion, the first sample chamber being defined by the first portion, the second portion defining a second sample chamber.

11. The biopsy system of claim 10, the pivot being positioned between the first portion and the second portion, the body being configured to rotate relative to the pivot to alternatingly move the first sample chamber and the second sample chamber between the sapling axis and the imaging axis.

12. The biopsy system of claim 8, the body defining a triangular cross-sectional shape.

13. The biopsy system of claim 8, the body defining a hourglass cross-sectional shape.

14. The biopsy system of claim 1, the tissue transport tube being configured to communicate one or more tissue samples therethrough using vacuum, saline, or a combination thereof.

15. The biopsy system of claim 1, further comprising an analysis assembly, the analysis assembly including an x-ray source and an x-ray detector, the x-ray source and the x-ray detector being positioned relative to each other to define the imaging axis.

16. An apparatus for use with a biopsy device, the apparatus comprising:
(a) a tissue transport tube, the tissue transport tube being configured to receive and transport one or more tissue samples acquired by the biopsy device;
(b) a tissue sample holder, the tissue sample holder including a tissue tray, the tissue tray being in selective communication with the tissue transport tube to receive the one or more tissue samples acquired by the biopsy device from the tissue transport tube; and
(c) an analysis assembly defining an imaging axis extending from a detector, the tissue tray being configured to move relative to the analysis assembly into alignment with the imaging axis to analyze the one or more tissue samples acquired by the biopsy device.

17. The apparatus of claim 16, the tissue transport tube defining a sampling axis, the sampling axis being offset relative to the imaging axis.

18. The apparatus of claim 16, further comprising a vacuum tube, the vacuum tube including a distal opening positioned opposite a proximal opening of the tissue transport tube, the vacuum tube being configured to receive one or more tissue samples from the tissue tray.

19. The apparatus of claim 16, the analysis assembly including a source and the detector, the source being an x-ray source, the detector being an x-ray detector.

20. A method of analyzing a tissue sample, the method comprising:
(a) transporting a tissue sample acquired by a biopsy device through a tissue transport tube into a sample chamber of a tissue tray aligned with a sampling axis defined by the tissue transport tube;
(b) moving the sample chamber of the tissue tray relative to the sampling axis to align the sample chamber with an imaging axis; and
(c) imaging the tissue sample while the sample chamber of the tissue tray is aligned with the sampling axis.

21.-24. (canceled)
